(19) United States
(12) Patent Application Publication
(10) Pub. No.: US 2010/0183119 A1
(43) Pub. Date: Jul. 22, 2010

(54) DIAGNOSTIC UNIT HAVING A PROTECTIVE DEVICE

(76) Inventors: Jasmina Ludwig, München (DE); Thomas Mertelmeier, Erlangen (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/664,942

(22) PCT Filed: Sep. 2, 2008
(86) PCT No.: PCT/EP08/61565

§ 371 Date: Dec. 16, 2009

(30) Foreign Application Priority Data
Oct. 8, 2007 (DE) 10 2007 048 165.0

(51) Int. Cl. A61B 6/10

(52) U.S. Cl. 378/37

(57) ABSTRACT

A radiography diagnostic apparatus has a radiation source that emits an x-ray beam, and a protection that is mounted to prevent unwanted body portions of a patient, or operating personnel, from interacting with the x-ray beam.

### BACKGROUND OF THE INVENTION

1. Field of the Invention

The invention concerns a diagnostic unit with a protective device for patients and/or operators.

2. Description of the Prior Art

A diagnostic unit (in particular a mammography apparatus or tomosynthesis apparatus) includes, among other things, a subject table on which a subject to be examined can be positioned as well as an x-ray radiator head that has at least one x-ray source. The x-ray radiator head is supported such that it can be rotated in a stand. The x-ray detector is located within or below the subject table. During the x-ray acquisition, the x-ray radiator head is positioned above the subject table. For x-ray acquisition, the patient is brought very close to the subject table. During an x-ray image acquisition, the patient stands or sits in front of the subject table of the diagnostic unit.

A mammography apparatus with a face protection integrated into an x-ray radiator head is disclosed in DE 10 2006 038 063 A1. Contact of the face of the patient with the x-ray beam emanating from the x-ray radiator head is prevented by this face protection.

### SUMMARY OF THE INVENTION

An object of the invention is to provide an additional protection device for a diagnostic unit.

This object is achieved in accordance with the invention by a diagnostic unit having a protective element that can be attached in a stationary manner to a subject fixed on the subject table.

The invention has the advantage that arbitrary position changes of the x-ray radiator head between the desired position and rest position can ensue in addition to a secure, limitation-free positioning of the patient, independent of the acquisition mode.

The invention also has the advantage that both a fixing of the subject to be examined and a spatial separation between the patient and the x-ray radiator head are achieved.

The invention has the further advantage that the patient does not come into contact with the rotating x-ray radiator head given a stereotactic biopsy in which two acquisitions are implemented with an offset angle, or a tomosynthesis in which an entire scan of a subject is acquired with multiple projections across a larger angular range.

The invention has the advantage that access to the subject is possible at any time for an intervention (for example biopsy) and for a correct patient positioning.

In an embodiment of the invention the size of the protective area of the protective element can be varied (for example by extending or unfolding) before the movement of the x-ray radiator head, in particular before a tomosynthesis.

In a further embodiment of the invention the protection shield is arranged so as to be movable in front of the x-ray head that is mounted such that it can rotate in a stand, and the protection shield can be shifted upwardly, downwardly or laterally and forms a stationary separation wall between patient and the moved x-ray head.

### BRIEF DESCRIPTION OF THE DRAWINGS

FIG. 1 schematically illustrates a mammography apparatus with face protection, as a first embodiment of the present invention.

FIGS. 2, 3 and 4 respectively illustrate further embodiments of a protective element installed in a diagnostic apparatus in accordance with the present invention.

### DESCRIPTION OF THE PREFERRED EMBODIMENTS

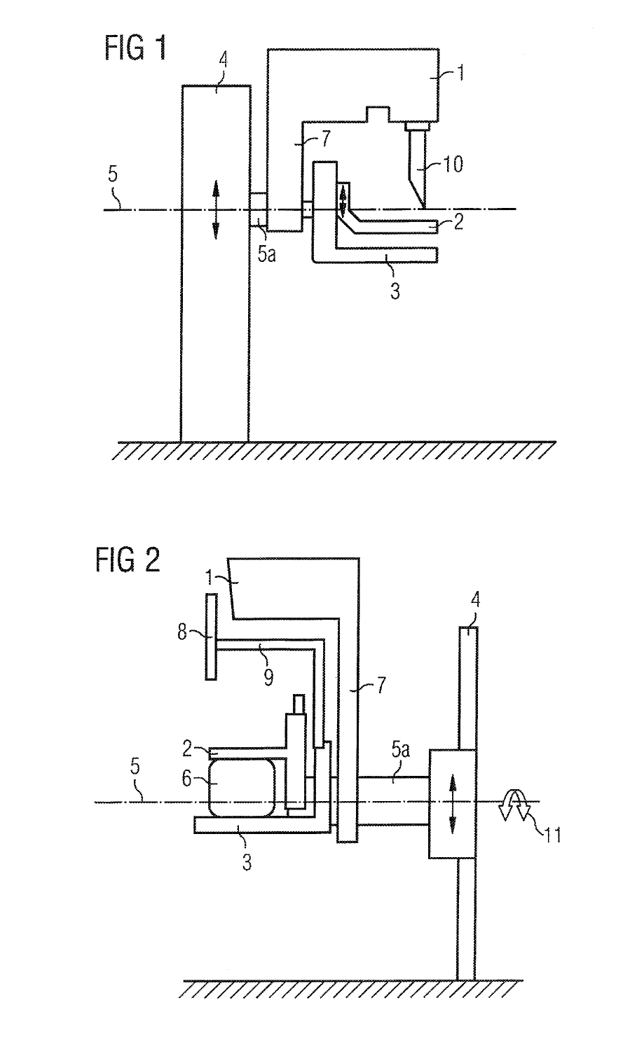

In FIG. 1 a mammography apparatus is depicted that has a face shield 11 for patients or operators that can be integrated into an x-ray radiator head 1. The x-ray radiator head 1 has (not shown in detail) an x-ray source together with diaphragm and filter. After traversing the diaphragm and filter, an x-ray beam generated by the x-ray source exits the radiator head through the x-ray beam aperture and strikes the subject table 3 after passing through the breast to be examined. An x-ray detector (likewise not shown in detail) is arranged within or below the subject table 3 and detects the incident x-ray radiation. The x-ray radiator head 1 is supported on a stand 4 with an acquisition unit 5a that can rotate around a horizontal axis 5. A subject table 3 is likewise arranged such that it can rotate around this horizontal axis 5. The x-ray radiator head 1 is connected with the acquisition unit 3 by a pivot frame 7. A compression plate 2 serves to fix the breast on the subject table 3. The face shield 10 is arranged directly at the beam output of the x-ray source of the x-ray radiator head 1. For an examination a patient is positioned near the subject table 3. The face shield 10 is fixed at the head height of the patient such that the x-ray beam escaping from the x-ray radiator head 1 does not arrive at the head of the patient.

A further arrangement of a diagnostic unit (in particular of a mammography apparatus or a tomosynthesis apparatus) with a protective element 8 according to the invention is schematically shown in FIG. 2. The x-ray radiator head 1 with a pivot frame 7 is horizontally fixed over an acquisition element 5a such that it can rotate in a stand 4. The fixable detector 3 and the likewise fixable and height-adjustable compression plate 2 are arranged on this rotatable acquisition element 5a. The rotation axis 5 is located at the level of the subject 6. In this embodiment, an attachment 9 for the protective element 8 is arranged on an extension of the fixable compression plate 3. An attachment to the height-adjustable compression plate 3 is likewise possible. In this embodiment, the protective element 8 is arranged stationary relative to the fixed subject 6 on the one hand and separate from the movable x-ray radiator head 1 on the other hand. The protective element 8 in the form of a protective shield produces a spatial, large-area separation between the patient and/or operator and the x-ray radiator head and thus enables a collision-free acquisition procedure. During an acquisition procedure (in particular during a tomosynthesis), the x-ray radiator head 1—moved manually or controlled by a program—can execute an x-ray acquisition from a predeterminable offset angle or an entire scan across a larger angular range with multiple projections. The patient positioned and aligned in front of the mammography or, respectively, tomosynthesis apparatus is thus spatially separated by the protective element 8 from the x-ray radiator head 1 executing a rotation movement 11 in the entire movement range of his upper body. In addition to the immediate protection for the patient, the protective element 8 also offers a task facilitation for the operator and an optimized workflow since an immediate supervision of the patient by the operator can be omitted, in particular during the x-ray acquisitions.

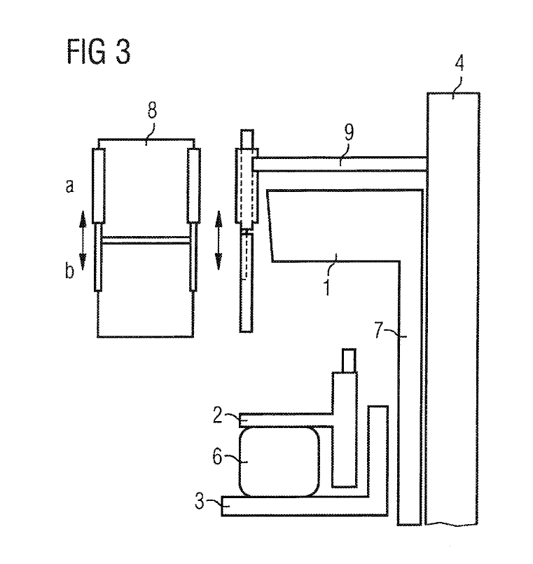

A further variant for the arrangement and embodiment of the protective element 8 at the mammography or tomosynthesis apparatus is shown in FIG. 3. In this embodiment the attachment 9 is attached to the stand 4. In this embodiment the protective element 8 is equipped so as to be height-adjustable. For example, the protective element 8 as shown in the side view and front view can be displaced between the regions a through b and be positioned for an effective patient protection corresponding to the requirements and needs during a session. In the rest state, the protective element 8 can remain at position 1. After the end of the examination or acquisition procedure, the protective element 8 can be displaced upward into a rest position. The retention device for the protective element 8 thus is not attached to the x-ray radiator head 1 but rather is attached to components of the apparatus so as to be fixed or, respectively, stationary relative to the patient.

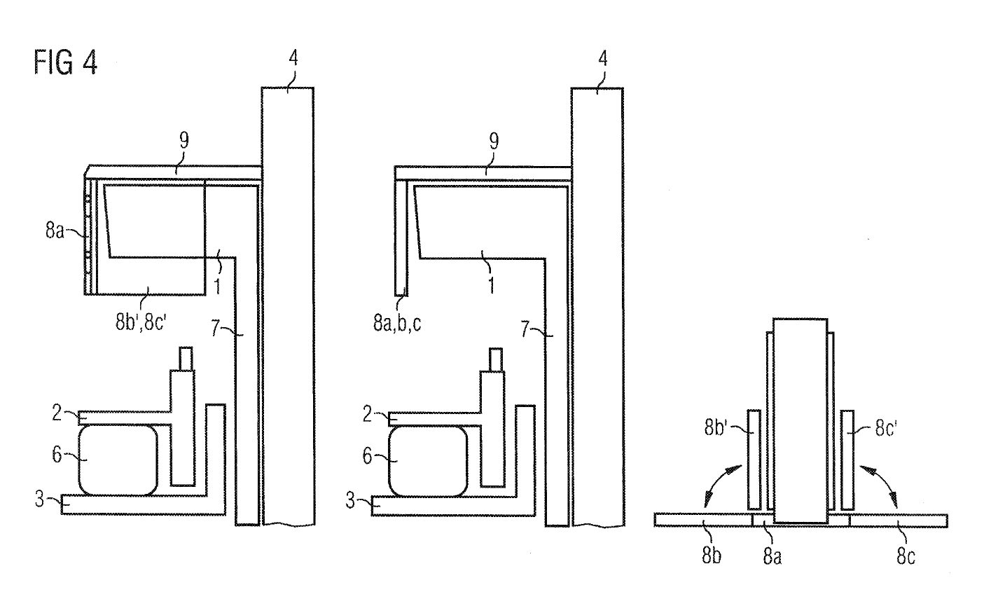

A further example of an arrangement of a design of a multipart protective element 8a, 8b, 8c is shown in FIG. 4, wherein this is attached by an attachment 9 to the stand 4 of the mammography apparatus. In this embodiment the middle part of the protective element 8 is connected with the respective blade elements via connection elements. The connection elements can be fashioned as hinges. The hinges respectively connect the parts 8a with 8b and 8a with 8c. The multipart protective element is held by means of an attachment means 9. This attachment 9 can span the x-ray radiator head in a flat, rod-like or semicircular shape. The attachment 9 is mounted on the stand 4 of the mammography apparatus. The attachment 9 is connected with side situated opposite the attachment side, for example with the upper edge of the middle first part of the protective element 8a. The attachment 9 has the necessary flexural strength in order to be allow no noticeable oscillation of the protective element upon contact and to keep this in a stable position. In the operating position, the partial surfaces 8b, 8c of the protective element 8 are folded out. In FIG. 4, the protective element 8 is depicted in operating position 8a, 8b and 8c (shown from above) and in a rest position 8a, 8b′ and 8c′ (here shown in a side view). In the rest position the partial surfaces 8b′, 8c of the protective shield are arranged parallel to the flanks of the x-ray radiator head 1. If the patient has been positioned on the patient or subject table 3 for an examination, the protective element 8 is folded in front of this. The protective element 8 in its unfolded state can also be fashioned so as to be height-adjustable or laterally adjustable. In addition to the described variation of the protective surface area and position of the protective element, this can also be modified via an unfolding or extension of protective elements stowed in the manner of a Venetian blind.

Although modifications and changes may be suggested by those skilled in the art, it is the intention of the inventors to embody within the patent warranted hereon all changes and modifications as reasonably and properly come within the scope of their contribution to the art.

We claim as our invention:

1-5. (canceled)

6. A radiography diagnostic apparatus comprising:
an x-ray radiator that radiates x-rays;
a patient table adapted to receive a patient thereon;
an x-radiator that detects the x-rays radiated by the x-radiator after passage through the examination subject on the patient table;
a stand that supports the x-ray radiator and permits rotation of the x-radiator relative to the patient table with the examination subject thereon and relative to the radiation detector;
a protective element having a size;
an attachment that attaches said protective element to a component selected from the group consisting of said stand and said patient table, at a position that prevents a body portion of the examination subject that is not to be irradiated by said x-rays from interacting with said x-ray beam; and
at least one of said size of said protective element and said position of said protective element being variable.

7. An apparatus as claimed in claim 6 wherein said patient table comprises a table extension, and wherein said protective element is attached to said extension.

8. An apparatus as claimed in claim 6 wherein said attachment allows height-adjustment of said protective element relative to said patient table.

9. An apparatus as claimed in claim 6 wherein said protective element is comprised of multiple parts configured to change said size of said protective element.
